(19) United States
(12) Patent Application Publication
(10) Pub. No.: US 2005/0209520 A1
(43) Pub. Date: Sep. 22, 2005

(54) Two-node-type human physiological parameter measuring apparatus

(75) Inventor: Chao-Chuan Chen, Taichung Hsien (TW)

Correspondence Address: BACON & THOMAS, PLLC, 625 SLATERS LANE, FOURTH FLOOR, ALEXANDRIA, VA, 22314, US

(73) Assignee: CHANG YOW INDUSTRY CO., LTD., CHANGHUA HSIEN (TW)

(21) Appl. No.: 10/923,734

(22) Filed: Aug. 24, 2004

(30) Foreign Application Priority Data
Mar. 16, 2004 (TW) 93208939

(51) Int. Cl. A61B 5/02; A61B 5/05

(52) U.S. Cl. 600/500; 600/547

(57) ABSTRACT

A two-node-type human physiological parameter measuring apparatus includes two pulse sensors each having a contact terminal formed at its external surface, two elastic clamp portions extending downwards from two sides of a bottom of the two pulse sensors, and a clamping space formed between the two clamp portions, two wires, and a central processor electrically connected via the two lead wires respectively with the pulse sensors for inputting and outputting the voltage signal and converting the same by a specific program into data output, for the measurement of heartbeat frequency or body fat quantity.

### BACKGROUND OF THE INVENTION

1. Field of the Invention

The present invention relates generally to human physiological parameter measuring apparatuses, and more particularly to a two-node-type human physiological parameter measuring apparatus.

2. Description of the Related Art

Human physiological parameter measuring apparatuses are now widely applied on various types of medical hygienic instruments and exercise apparatuses. The human physiological parameter measuring apparatus is generally operated by that mount a pulse sensor on a predetermined position of the instrument or apparatus, and then the trier has the hands respectively touch the pulse sensor to proceed with measuring the human physiological parameters, such as heartbeat frequency, body fat rate, etc.

In addition, the conventional human physiological parameter measuring apparatus is usually four-node-typed, having two contact terminals mounted inside one pulse sensor. Each of the pulse sensors must have at least two lead wires connected therewith, such that the layout of the lead wires is complicated in consideration of insulation preventing the sensors from incorrect measurement caused by signal interference generated by short circuits between the multiple lead wires. In addition, to avoid short circuits during the manufacturing process, guide slots for receiving the lead wires must be additionally formed and insulation layer must be increased more to prevent the lead wires from short circuits caused by accidental contact while mounting the lead wires, however postponing the time schedule of processing to increase the manufacturing cost.

Therefore, the applicant invented two kinds of two-node-type human physiological parameter measuring apparatuses. One is the two-node-type heartbeat frequency measuring apparatus (patent pending), which touches human body, usually hand or foot, by means of two contact terminals thereof to detect human pulse signal to further access the human heartbeat frequency. The other is the two-node-type human fat measuring apparatus (Taiwan Patent Publication No. 454501), which touches human body by means of two contact terminals thereof to detect human capacitive parameters to further access the human fat. However, when the trier is operating any of the aforementioned measuring apparatuses, the trier is still confined by where the sensors of the measuring apparatus are located and fails to operate the measuring apparatus with a comfortable posture subject to the trier's favor, such that the imperfect contact may be incurred between the trier and the sensors due to the trier's improper posture.

### SUMMARY OF THE INVENTION

The primary objective of the present invention is to provide a two-node-type human physiological parameter measuring apparatus, which pulse sensors each have only one contact terminal connected to a central processor via a lead wire to dramatically simplify the wire layout.

The secondary objective of the present invention is to provide a two-node-type human physiological parameter measuring apparatus, which is appropriately located at where the trier can comfortably touch and hold the apparatus to prevent the measurement from error due to imperfect contact incurred by the trier's improper posture.

To achieve the foregoing objectives, the present invention provides the two-node-type human physiological parameter measuring apparatus, which is comprised of two pulse sensors each have a contact terminal provided on an external surface thereof, two elastic clamp portions extending downwards from two sides of a bottom thereof, and a clamping space formed between the two clamp portions, two lead wires, and a central processor electrically connected via the two wires respectively with the pulse sensors for receiving and converting voltage signals by a specific program into data output.

### BRIEF DESCRIPTION OF THE DRAWINGS

FIG. 1 is a schematic view of a first preferred embodiment of the present invention;

FIG. 2 is a perspective view of a pulse sensor of the first preferred embodiment of the present invention;

FIG. 3 is a perspective view of the first preferred embodiment of the present invention, showing that the pulse sensor is clamped to a bar;

FIG. 4 is a perspective view of a pulse sensor of a second preferred embodiment of the present invention; and

FIG. 5 is a perspective view of a pulse sensor of a third preferred embodiment of the present invention.

### DETAILED DESCRIPTION OF THE INVENTION

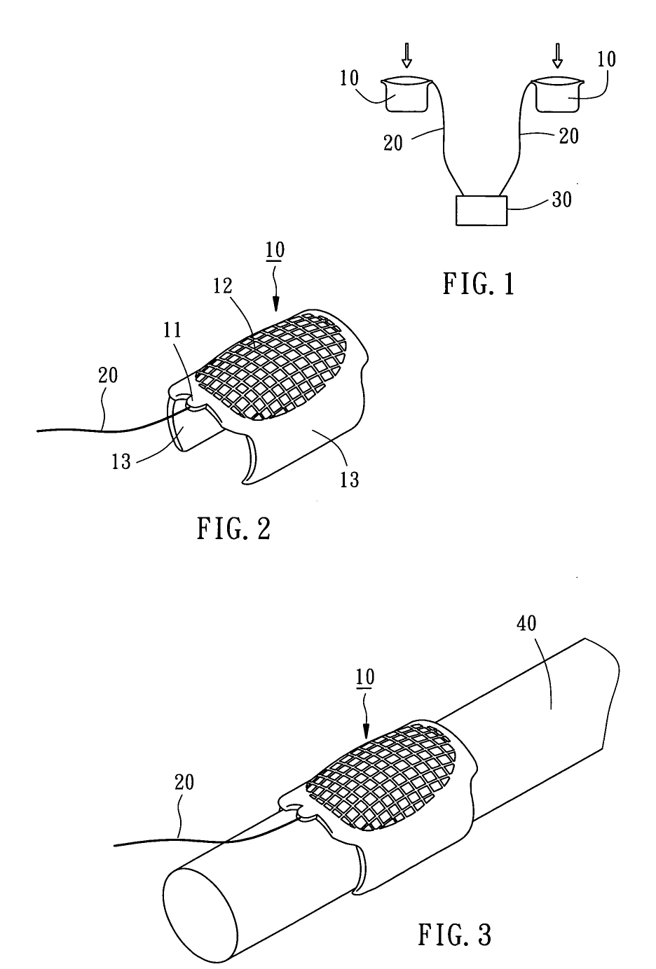

Referring to FIGS. 1 and 3, a two-node-type human physiological parameter measuring apparatus constructed according to a first preferred embodiment of the present invention is comprised of two elongated pulse sensors 10, two lead wires 20, and a central processor 30. Each of the pulse sensors 10 is formed in one-piece of electrically conductive metal, including a conductive point 11, a convex top surface 12, and two elastic clamp portions 13. The conductive point 11 is connected with the lead wire 20 by welding. The top surface 12 defines a contact terminal for touching by the trier's hand, being reticulated to have a plurality of apertures for air-permeable and skidproof purposes to prevent the trier's hand from slipping off the pulse sensor 10 due to perspiration. The two clamp portions 13 respectively extend downwards from bilateral sides of the pulse sensor 10, and a clamping space is defined between the two clamp portions 13. The two elastic clamp portions 13 have resilience generated while stretched by a force to further clamp the pulse sensor 10 to an electrically insulated bar 40. The trier can clamp the pulse sensor 10 to a proper position of the bar 40 and adjustably move the pulse sensor 10 to where the trier feels comfortable for holding and touching. The central processor 30 is electrically connected via the two lead wires 20 respectively with the pulse sensors 10 for receiving and converting voltage signals by a specific program into data output.

As shown in FIG. 1, when the trier touches and holds the two pulse sensors 10 respectively by the hands or feet, a circuit loop is formed together by the trier, the two pulse sensors 10, the two lead wires 20, and the central processor 30, and then the central processor 30 obtains the voltage signal from the sensors 10 and analyzes the same to output the data for accessing the desired human physiological parameter. For instance, the heartbeat frequency can be accessed by scanning the human potential difference; the human fat quantity can be accessed by scanning the human capacitive parameter or resistance.

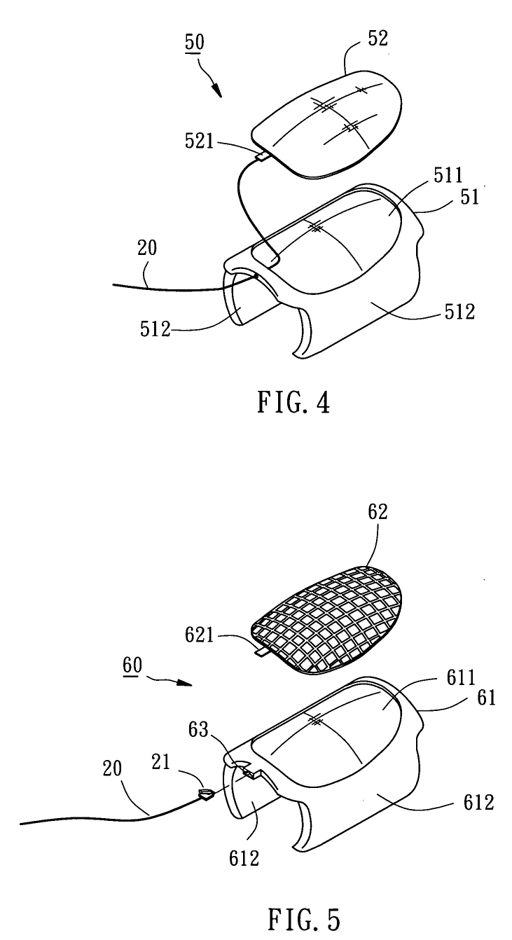

Referring to FIG. 4, the two-node-type human physiological parameter measuring apparatus constructed according to a second preferred embodiment of the present invention is similar to the first preferred embodiment but different only by the following recitation.

Each of the pulse sensors 50 is comprised of an elongated detecting member 51 and an electrically conductive member 52. The detecting member 51 is made of electrically insulated plastic material, including a convex top surface, a concave portion 511, and two elastic clamp portions 512 extending downwards from two elongated sides thereof. A clamping space is defined between the two clamp portions 512. The two elastic clamp portions 512 are elastically deformable for generating the resilience while stretched to clamp the pulse sensor 50 to an electrically insulated bar 40. Accordingly, the trier can adjustably clamp the pulse sensors 50 to where the trier feels comfortable to hold and touch the bar 40. The electrically conductive member 52 is made of a metallic piece and inserted into the concave portion 511 to be engaged with the top surface of the detecting member 51, thereby defining a contact terminal for holding and touching by the trier. The electrically conductive member 52 has a conductive point 521. Each of the lead wires 20 is inserted into the detecting member 51 to be connected with the conductive point 521 by welding.

Referring to FIG. 5, the two-node-type human physiological parameter measuring apparatus constructed according to a third preferred embodiment of the present invention is similar to the second preferred embodiment but different only by the following recitation.

Each of the pulse sensors 60 is comprised of an elongated electrically insulated detecting member 61 and an electrically conductive member 62. The detecting member 61 is provided with a socket 63 protruded therefrom. The electrically conductive member 62 has a reticulated metallic piece inserted into the concave portion 611 to be engaged with the top surface 61, thereby defining a contact terminal for trier's holding and touching. The electrically conductive member 62 has a conductive point 621 connected to the socket 63. Each of the lead wires 20 has a plug 21 detachably connected with the socket 63, such that the two lead wires 20 can be detachably interconnected respectively with the two electrically conductive members 62.

Each of the electrically conductive member mentioned in the second and third embodiments of the present invention can be alternatively made of, in addition to a metallic piece, an electrically conductive film or other electrically conductive material, of which the trier feels different touch on the hands for the trier's diversified options.

The advantages of the present invention are as recited thereafter. Each of the pulse sensors includes only one contact terminal connected with the central processor by merely one lead wire, such that the wire layout is dramatically simplified to reduce the production cost of the additional arrangement of the guide slots and to prevent short circuits and interference incurred by imperfect insulation due to the overcrowding of the excessive lead wires inside the pulse sensors. In addition, the pulse sensors are movably mounted to where the trier feels comfortable for holding and touching it to avoid the imperfect contact incurred by improper holding posture.

1. A two-node-type human physiological parameter measuring apparatus comprising:
two pulse sensors each formed in one-piece of an electrically conductive material to define a contact terminal for handling by the trier's hands, said pulse sensor having two elastic clamp portions extending downwards from two sides thereof, a clamping space being formed between said two clamp portions;
two lead wires; and
a central processor electrically connected via said two lead wires respectively with said two pulse sensors for receiving and converting voltage signal detected between said two pulse sensors, computing the voltage signal by a specific program, and then outputting data of the computation.

2. The two-node-type human physiological parameter measuring apparatus as defined in claim 1, wherein each of said pulse sensors comprises a reticulated top surface having a plurality of apertures.

3. The two-node-type human physiological parameter measuring apparatus as defined in claim 1, wherein each of said pulse sensors comprises a conductive point connected with said lead wire by welding.

4. The two-node-type human physiological parameter measuring apparatus as defined in claim 1, wherein said pulse sensor further comprises a socket protruded therefrom; each of said lead wires further comprises a plug provided at a front end thereof and detachably connected with said socket of said pulse sensor.

5. The two-node-type human physiological parameter measuring apparatus comprising:
two pulse sensors each having a detecting member and an electrically conductive member, said detecting member having a concave portion on its top surface, said electrically conductive member being inserted into said concave portion to be engaged with said top surface of said detecting member, said detecting member having two elastic clamp portions extending downwards from two sides of its bottom, a clamping space being formed between said two clamp portions;
two lead wires; and
a central processor electrically connected via said two lead wires with said electrically conductive members for receiving and converting voltage signal detected between said two electrically conductive members, computing the voltage signal by a specific program, and then outputting data of the computation.

6. The two-node-type human physiological parameter measuring apparatus as defined in claim 5, wherein each of said electrically conductive members comprises a conductive point connected to said lead wire by welding.

7. The two-node-type human physiological parameter measuring apparatus as defined in claim 5, wherein said detecting member further comprises a socket protruded therefrom; said electrically conductive member comprises a conductive point connected with said socket of said detecting member, each of said two lead wires having a plug provided at its front end and detachably connected to said socket.

8. The two-node-type human physiological parameter measuring apparatus as defined in claim 5, wherein each of said electrically conductive members is a metallic piece.

9. The two-node-type human physiological parameter measuring apparatus as defined in claim 5, wherein each of said electrically conductive members is a reticulated metallic piece.

10. The two-node-type human physiological parameter measuring apparatus as defined in claim 5, wherein each of said electrically conductive members is an electrically conductive film.
